(19) United States
(12) Patent Application Publication
(10) Pub. No.: US 2026/0130754 A1
(43) Pub. Date: May 14, 2026

(54) Syndesmosis Fixation System

(71) Applicant: Acuitive Technologies, Inc., Allendale, NJ (US)

(72) Inventors: Rui J. Ferreira, Livingston, NJ (US); Cesar Chavista, Rahway, NJ (US); Wayne Berberian, Rahway, NJ (US); Ryan Andrews, Elmer, NJ (US)

(73) Assignee: Acuitive Technologies, Inc., Allendale, NJ (US)

(21) Appl. No.: 19/389,830

(22) Filed: Nov. 14, 2025

Related U.S. Application Data
(60) Provisional application No. 63/720,398, filed on Nov. 14, 2024.

(51) Int. Cl. A61F 2/08; A61B 17/04

(52) U.S. Cl. CPC A61F 2/0811; A61B 17/0401; A61B 2017/0404; A61B 2017/0409; A61B 2017/0427; A61B 2017/0464; A61F 2002/0829; A61F 2002/0852; A61F 2002/0882

(57) ABSTRACT

The present disclosure relates to systems and methods for treatment using syndesmosis fixation. More specifically, syndesmosis fixation is provided with physical features produced from a citrate-based composite biomaterial to aid in healing of the syndesmotic ligament.

### CROSS-REFERENCE TO RELATED APPLICATION

The present application claims priority benefit to a U.S. provisional patent application entitled “Syndesmosis Fixation System,” which was filed on Nov. 14, 2024, and assigned Ser. No. 63/720,398. The entire content of the foregoing U.S. provisional patent application is incorporated herein by reference.

### TECHNICAL FIELD

The present disclosure relates to systems and methods for treatment using syndesmosis fixation. More specifically, syndesmosis fixation is provided with physical features produced from a citrate-based composite biomaterial to aid in healing of the syndesmotic ligament.

### BACKGROUND

A syndesmosis injury, also known as a high ankle sprain, is a tear or damage to the ligaments in the fibrous joint (that can occur through high ankle sprain or fracture). Many times, the syndesmotic injury is repaired by using rigid fixation, such as screws or dynamic fixation, such as suspensory buttons. The rigid fixation typically, if left in the patient after healing, leads to a high rate of fracture since the screw is placed through the fibula and into the tibia to allow the syndesmotic ligament to heal. However, since these two bones have relative motion between them during a normal gait cycle, this relative motion can ultimately lead to fatigue fracture of the screws. The fracture can occur anywhere along the length of the screw, making removal of the broken screw fragments complex and difficult. Additionally, there is not a lot of soft tissue surrounding the tibia and fibula leading to discomfort when using a rigid fixation.

Dynamic fixation is another method of stabilization that typically uses two titanium or stainless-steel buttons or toggles that are positioned on the outer cortex of the fibula and tibia and are connected via a suture. This type of configuration requires a bore hole be drilled through the tibia and the fibula and compresses the space between the tibia and fibula, thereby maintaining bone stabilization with minimal motion between the bones so as to allow the syndesmotic ligament to heal during the normal gait cycle. However, such a procedure can be time consuming and difficult to perform. For example, when drilling a bore hole through the tibia and the fibula, the surgeon must take care not to damage any blood vessels or nerves on the opposite side of the tibial bone. Additionally, the toggle bolt or suture buttons anchored on the cortex may create protrusions or “bumps” on the bony surface that can irritate the patient's skin. If the suture is too tight, the toggle bolt or suture buttons may become embedded in the bone and, if the suture is too lose, then there is no stabilizing and the syndesmotic ligament will not heal properly.

Accordingly, there is a need for a device and method of use which addresses the aforementioned shortcomings.

This section is intended to introduce the reader to various aspects of art that may be related to various aspects of the present disclosure described or claimed below. This description is believed to be helpful in providing the reader with background information to facilitate a better understanding of the various aspects of the present disclosure. Accordingly, it should be understood that these statements are to be read in this light and not as admissions of prior art.

### SUMMARY

In one aspect, a syndesmosis fixation device is provided. The syndesmosis fixation device includes a bone anchor having a plurality of barbs and a button operably connected to the bone anchor by one or more sutures. The one or more sutures are actuatable to bias the button towards the bone anchor. The syndesmosis fixation device is inserted into a bone tunnel through the fibula and into the tibia, such that the bone anchor is fixed within the tibia. The button is biased against the fibula and towards the bone anchor to stabilize the tibia and fibula, thereby allowing a syndesmosis injury to heal.

In exemplary embodiments, a biodegradable citrate-based suture button and suture anchor design are provided to approximate and retain two bone segments relative to each other with eventual resorption of the implants and regeneration of the tissues.

Various refinements of the features noted in relation to the above-mentioned aspects exist. Further features may also be incorporated in the above-mentioned aspects as well. These refinements and additional features may exist individually or in any combination. For instance, various features discussed below in relation to any of the illustrated examples may be incorporated into any of the above-described aspects, alone or in any combination.

### BRIEF DESCRIPTION OF DRAWINGS

The subject matter, which is regarded as the disclosure, is particularly pointed out in the specification. The foregoing and other features, and advantages of the disclosure are apparent from the following detailed description taken in conjunction with the accompanying drawings in which like reference numerals refer to like elements throughout the different views:

FIG. 1 is a schematic view of a syndesmosis fixation device with two suture loops;

FIGS. 2A-2B are schematic views of bone anchors having different barb configurations;

FIGS. 3A-3C are schematic views of a syndesmosis fixation device with two suture loops being tightened;

FIGS. 4A-4B are schematic views of a syndesmosis fixation device with one suture loop;

FIG. 5 is a perspective view of a syndesmosis fixation device being inserted into a patient's ankle;

FIGS. 6A-6B are perspective views of two syndesmosis fixation devices with sponge devices inserted into a patient's ankle;

FIG. 7 is a flow chart of a method of treatment using a syndesmosis fixation device.

### DETAILED DESCRIPTION

The following detailed description and examples set forth preferred materials, components, and procedures used in accordance with the present disclosure. This description and these examples, however, are provided by way of illustration only, and nothing therein shall be deemed to be a limitation upon the overall scope of the present disclosure.

The subject matter disclosed herein relates to syndesmosis fixation devices and methods of using syndesmosis fixation devices to treat syndesmosis injuries. More specifically, the present disclosure relates to an easy to install syndesmosis fixation devices made of a citric acid-based polymer which promotes soft tissue growth and integrates with a patient's bone.

In some embodiments, the disclosed syndesmosis fixation device includes a bone anchor having a plurality of barbs and a button operably connected to the bone anchor by one or more sutures. The one or multiple sutures are actuable to bias the button towards the bone anchor. The syndesmosis fixation device is inserted into a bone tunnel through the fibula and into the tibia such that the bone anchor is fixed to the tibia. The button is biased against the fibula and towards the bone anchor to fix the tibia and fibula in place, thereby allowing a syndesmosis injury to heal.

### Syndesmosis Fixation Device

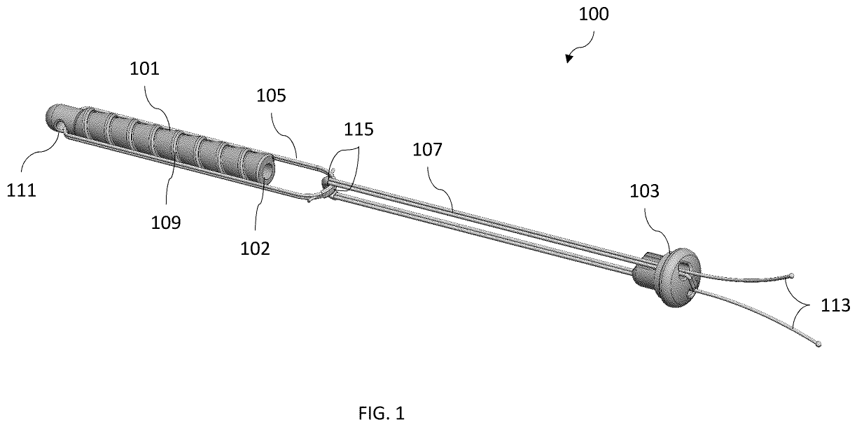

Referring now to FIG. 1, shown is a syndesmosis fixation device 100 according to some embodiments of the present disclosure. The syndesmosis fixation device 100 may include a bone anchor 101 and a button 103 connected by one or more suture loops 105, 107. The bone anchor 101 may be a cylindrical or generally cylindrical post with one or more barbs 109 thereon. The button 103 may be of various shapes, sizes, and diameters as desired to provide an anatomical fit to the bone. For example, in some embodiments, the button 103 may be a trauma plate. In some embodiments, use of a trauma plate may be desirable to fix a bone fracture or to reduce implant subsidence. In some embodiments, one or more bone anchors 101 may be operatively connected to the trauma plate.

In some embodiments, the bone anchor 101 and the button 103 may be made of a citric acid-based polymer. The citric acid-based polymer may be composited with ceramic constituents that elutes citrate, a key metabolite in the Krebs Cycle producing cell energy, to form a bio-composite material. This bio-composite material can be produced utilizing multiple/various manufacturing methods. The citrate polymer may possess various types of bio-ceramic fillers such as Bioglass, HA, TCP, calcium sulfate or various other ceramic fillers in either particle or fiber forms. The bio-ceramic filler can also be blends or mixes of the above-mentioned materials in various concentrations to match the clinical requirement of the procedure being addressed with the percent filler ranging from 0% to 70% by volume, although not limited thereto. In another embodiment, the bio-composite material can also be loaded and/or conjugated with various proteins, peptides or other small molecules to increase cellular response.

In some embodiments, the use of a citrate-based polymer used in both the bone anchor 101 and the button 103 incorporates design features that maintain mechanical stability while allowing the polymer composite to integrate into bone and participate in the soft tissue healing phase of the syndesmosis. This may allow for improved natural healing for patients. Additionally, allowing the polymer composite to integrate into the patient's own bone may also alleviate the need for potential revision surgery to remove broken hardware in the future.

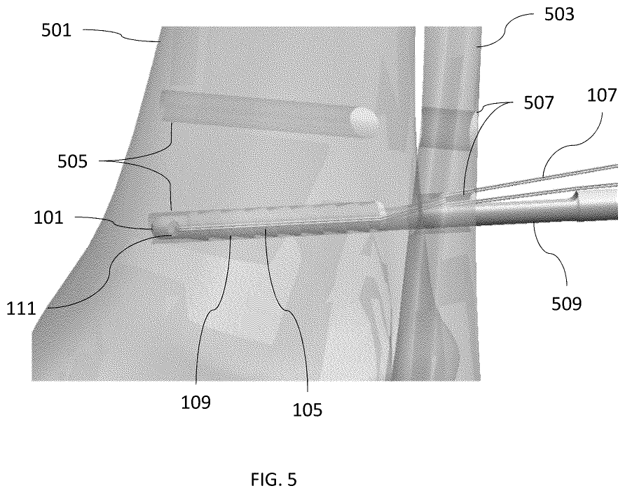

In some embodiments, the barbs 109 on the bone anchor 101 may be spiral configurations, for example single spirals, double concentric spirals, or double alternating spirals, discrete circular rings or diamond shaped protrusions, although not limited thereto. The barbs 109 may retain the bone anchor 101 in a first bone, for example the tibia. In some embodiments, the bone anchor 101 may be threaded and screwed into a first bone, for example the tibia, to retain the bone anchor 101 in the first bone. In some embodiments, the bone anchor 101 may have an inserter hole 102 on an end of the bone anchor 101. The inserter hole 102 is configured to receive a post of an inserter (shown in FIG. 5) to facilitate insertion of the bone anchor 101 into a first bone, for example the tibia.

A suture may be passed through a retaining hole 111 in the bone anchor 101 to form the anchor suture loop 105 and a button suture loop 107 passes through the anchor suture loop 105 and button 103 to operatively connect the bone anchor 101 and the button 103. The button suture loop 107 may be configured in such a pattern that the button suture loop 107 can be adjusted in size and be made smaller and approximate the button 103 towards the bone anchor 101 by pulling on the ends 113 of the button suture loop 107. In some embodiments, the button suture loop 107 may be pre knotted in several different configurations in such a manner that the button suture loop 107 self-locks as it is tightened. In this way, a user does not need to form a knot in the button suture loop 107 to maintain the compression formed by tightening the button suture loop 107 into a second smaller position.

In some embodiments, the retaining hole 111 may be on an end of the bone anchor 101 distal to the button 103 and opposite the inserter hole 102. In this way, when the bone anchor 101 is inserted into the bone, the anchor suture loop 105 is disposed along the bone anchor 101 and the barbs 109 and pressed into the bone. In some embodiments, it may be desirable to have the anchor suture loop 105 disposed along the bone anchor 101 and barbs 109, since the suture lines will increase the diameter of the bone anchor 101 making the press fit connection with the bone tighter.

In some embodiments, the suture forming the button suture loop 107 may be configured as a self-locking cinch. A helically wound braided suture is compressed separating the braided strands of the suture to create a gap, the ends 113 of the suture are passed though the gap creating a one-way self-locking cinch that tightens the button suture loop 107 as the ends 113 of the suture are pulled. The helically wound braid of the suture allows the ends 113 to be pulled through the gap in a first direction, tightening the loop, but seizes around the suture when attempting to move the ends 113 in a second reverse direction.

In some embodiments, the suture forming the anchor suture loop 105 may be double looped, passing through the retaining hole 111 twice, forming a double loop with two suture strands of the anchor suture loop 105 on either side of the bone anchor 101. In some embodiments, the suture forming the button suture loop 107 may be double looped, passing through the button 103 and the anchor suture loop 105 twice, forming a double loop with four suture strands (two pairs of strands) connecting the anchor suture loop 105 and the button 103. A double looped configuration of one or both anchor suture loop 105 and the button suture loop 107 may be desirable as the tensile force on the anchor suture loop 105 and the button suture loop 107 is distributed over multiple suture strands allowing for a greater compression force between the bone anchor 101 and the button 103.

In some embodiments, a sleeve 115 may be disposed around the anchor suture loop 105, the button suture loop 107, or a portion thereof. In some embodiments, the sleeve 115 may be used to reinforce or conceal a knot in the anchor suture loop 105 or the button suture loop 107. In some embodiments, the sleeve 115 may be positioned on the anchor suture loop 105 and the button suture loop 107 at the connection point between them to reinforce and prevent the suture loops 105, 107 from crimping.

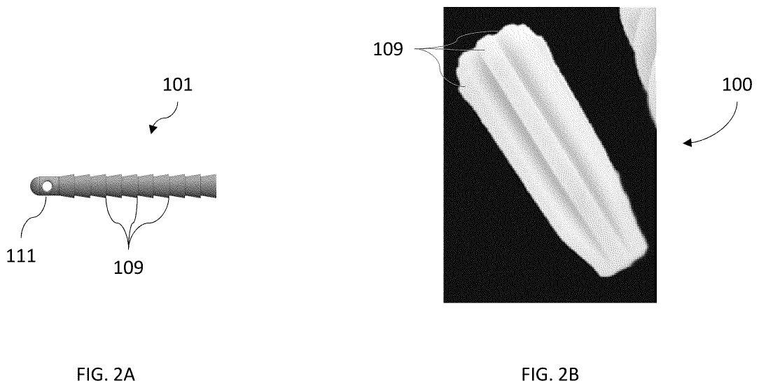

Referring now to FIGS. 2A-2B, shown are bone anchors 101 with various barb 109 configurations, according to some embodiments of the present disclosure. As illustrated in FIG. 2A, a bone anchor 101 with discrete circular ring barbs 109 is shown. As illustrated in FIG. 2B., a bone anchor 101 with diamond shaped protrusion barbs 109 is shown. In some embodiments, different configurations of the barbs 109 may provide different benefit. For example, spiral cut barbs 109 may be easier to manufacture.

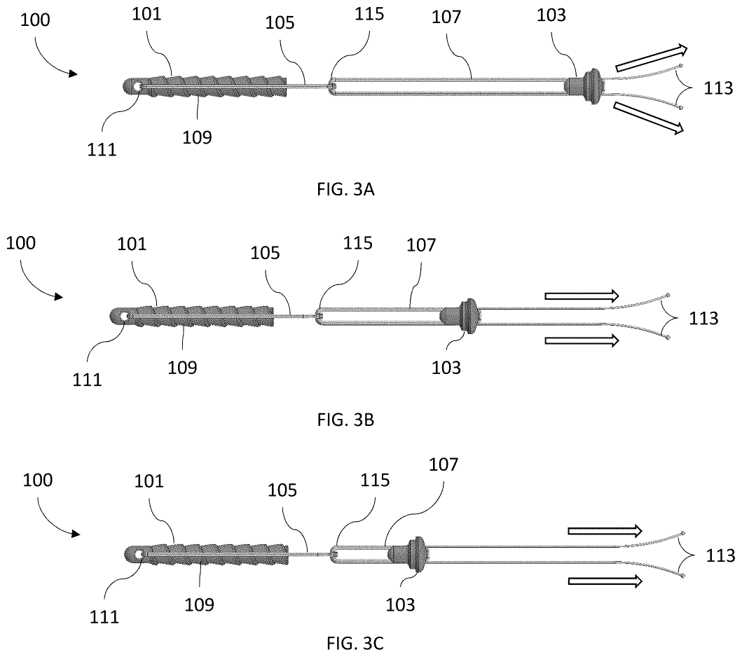

Referring now to FIGS. 3A-3C, shown is a button suture loop 107 of a syndesmosis fixation device 100 being tightened, according to some embodiments of the present disclosure. As illustrated in FIG. 3A, the button suture loop 107 is in a loose expanded position. As the ends 113 of the suture are pulled in the direction of the arrows, the button suture loop 107 is tightened, pulling the button 103 closer to the bone anchor 101. As illustrated in FIGS. 3B-3C, the button suture loop 107 becomes progressively tighter as the ends 113 of the suture are pulled.

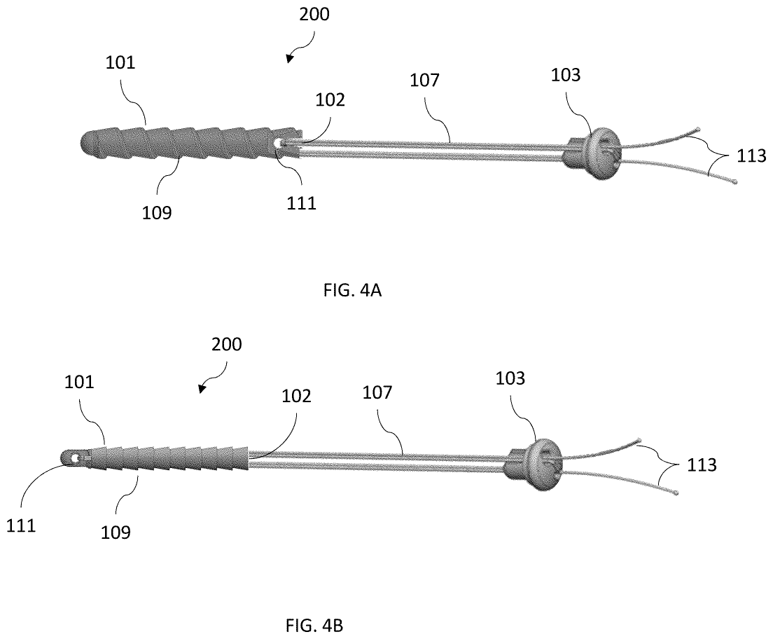

Referring now to FIGS. 4A-4B, shown is a syndesmosis fixation device 200 with a single suture loop, according to some embodiments. The syndesmosis fixation device 200 may be similar to the syndesmosis fixation device 100 except that a single suture loop, e.g., the button suture loop 107, is used as opposed to both an anchor suture loop 105 and button suture loop 107. As illustrated in FIG. 4A the retaining hole 111 is on an end of the bone anchor 101 proximal to the button 103. By changing the attachment point, i.e., the retaining hole 111, to be on an end of the bone anchor 101 proximal to the button 103, the syndesmosis fixation device 200 only requires a single suture loop, i.e., the button suture loop 107. This is because when tightening the button suture loop 107, the individual suture strands slide past each other. If the suture strands are pinned, for example between the bone anchor 101 and the bone, the button suture loop 107 cannot be tightened.

In some embodiments, the bone anchor 101 may have a cannulated hole through the entire or through substantially the entire body of the bone anchor 101. As illustrated in FIG. 4B the retaining hole 111 is on an end of the bone anchor distal to the button 103. The button suture loop 107 may be disposed through the cannulated hole and operatively connect the bone anchor 101 and the button 103. The suture strands of the button suture loop 107 may be freely slidable through the cannulated hole when the bone anchor 101 is inserted into a bone, since the suture strands are internal to the bone anchor 101 and not pinned against the bone. In this way, the button suture loop 107 may be tightened to bias the button 103 towards the bone anchor 101. In some embodiments, the cannulated hole may extend from the inserter hole 102.

### Syndesmosis Fixation Procedure

Referring now to FIG. 5, shown is the bone anchor 101 of a syndesmosis fixation device 100 being inserted into a tibia, according to some embodiments of the present disclosure. In some embodiments, the syndesmosis fixation device 100 may be used to fix two or more bones together when healing a syndesmosis injury, for example a tibia 501 and a fibula 503 when healing a high ankle sprain.

In some embodiments, a tibial tunnel 505 and a corresponding fibular tunnel 507 may be made in a user's respective tibia 501 and fibula 503 to receive the syndesmosis fixation device 100. In some embodiments, the tibial tunnel 505 may have a first diameter, being sized and dimensioned to receive the bone anchor 101 to form a tight press to fit connection, and the fibular tunnel 507 may have a second diameter, being sized and dimensioned to allow the bone anchor 101 but not the button 103 to be inserted through the fibular tunnel 507. This configuration of the tibial tunnel 505 and the fibular tunnel 507 may be desirable since the tibia 501 is larger than the fibula 503 and is better adapted for retaining the bone anchor 101.

An inserter 509 may be used to insert the bone anchor 101 through the fibular tunnel 507 into the tibial tunnel 505. In some embodiments, a post on the inserter 509 is slotted into the inserter hole 102 on the bone anchor 101 such that the bone anchor 101 acts as an extension of the inserter 509. The bone anchor 101 may then be inserted through the fibular tunnel 507 and into the tibial tunnel 505. The anchor suture loop 105 and the barbs 109 are pressed against the walls of the tibial tunnel 505 preventing the bone anchor 101 from being removed. The anchor suture loop 105 and the button suture loop 107 are loose and flexible and can be moved out of the way when the bone anchor 101 is inserted.

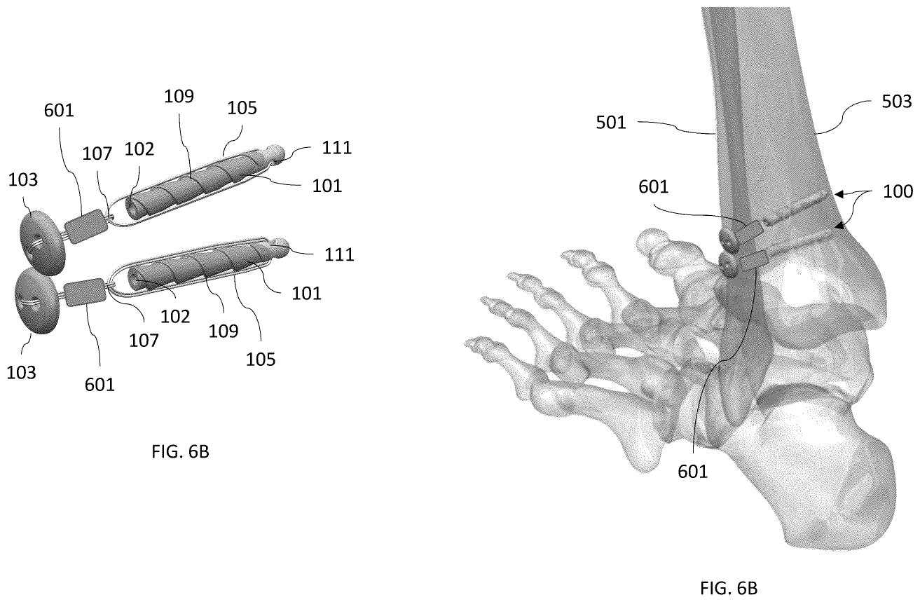

Referring now to FIGS. 6A-6B, shown is a syndesmosis fixation device 100 with a citrate-based graft, according to some embodiments of the present disclosure. In some embodiments, a synthetic citrate-based graft that acts as a scaffold to prevent scar tissue formation and participates in healing the interosseous ligament may be used in conjunction with a syndesmosis fixation device 100, 200. After preparing the bone tunnels in the fibula 503 and tibia 501, a cylindrical porous sponge 601 may be placed on the button suture loop 107 between the button 103 and the bone anchor 101 and pulled thru the fibular tunnel 507 when advancing the bone anchor 101 into the tibial tunnel 505. The synthetic (sponge-like) graft 601 may fill the fibular tunnel 507 and extrude beyond the fibula medial cortex into the interosseous space between the fibula 503 and tibia 501 eluting citrate to aid in healing the damaged syndesmotic membrane.

In some embodiments, one or more syndesmosis fixation devices 100 may be used to heal a syndesmosis injury. Additional syndesmosis fixation devices 100 will distribute the load across a wider area and may be desirable depending on the injury or the size of the user. As illustrated in FIG. 6B, two syndesmosis fixation devices 100 are used to fix the tibia 501 to the fibula 503.

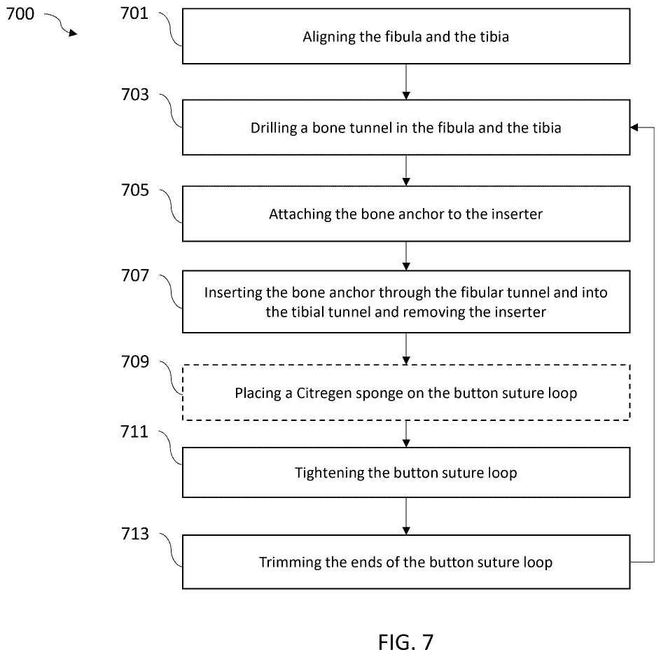

Referring now to FIG. 7, shown is a method 700 for treating a syndesmosis injury using a syndesmosis fixation device 100, according to some embodiments of the present disclosure. At 701 the procedure begins with a user, for example a physician or surgeon, aligning and fixing together the tibia 501 and the fibula 503 of a patient. The tibia 501 and the fibula 503 need to be aligned such that once the syndesmosis fixation device 100 is inserted, the tibia 501 and the fibula 503 are in the properly aligned rotational position for the syndesmosis injury to heal. In some embodiments, a jig may be used to hold the tibia 501 and the fibula 503 in place. In some embodiments, the jig may be left on during the entire procedure.

At 703, after drilling a fibular tunnel 507 in the fibula 503 and a tibial tunnel 505 in the tibia 501. In some embodiments, the fibular tunnel 507 has a greater diameter than the tibial tunnel 505. In some embodiments, a first drill bit is used to drill a hole through the fibula 503 to drill the tibial tunnel 505 and a second drill bit is used to drill the fibular tunnel 507. In some embodiments, a counter bored hole is drilled in the fibula 503 to recess the button 103 flush with the fibula 503. In some embodiments, a single drill bit is used to drill the tibial tunnel 505 and the fibular tunnel 507. The drill bit being sized and dimensioned to drill all the features of the tibial tunnel 505 and the fibular tunnel 507 at once.

After the tibial tunnel 505 and the fibular tunnel 507 are drilled, at 705, the bone anchor 101 is removably attached to the inserter 509 via the inserter hole 102 in the bone anchor 101. The anchor suture loop 105 and the button suture loop 107 are flexible and moved to the side to allow the inserter 509 to be attached to the bone anchor 101. In some embodiments, the inserter 509 is a wand acting as an extension of the bone anchor 101 to facilitate insertion of the bone anchor 101 through the fibular tunnel 507 and into the tibial tunnel 505. At 707, the bone anchor 101 is inserted through the fibular tunnel 507 and into the tibial tunnel 505. As the bone anchor 101 is inserted into the tibial tunnel 505, the barbs 109 and the anchor suture loop 105 are pressed against the walls of the tibial tunnel 505, securing the bone anchor 101 to the tibia 501. The inserter 509 is then removed. Since the bone anchor 101 is secured to the tibia 501, when the inserter 509 is pulled back, the inserter 509 is disengaged from the inserter hole 102 leaving the bone anchor 101 in the tibial tunnel 505.

In some embodiments, at 709, a resorbable synthetic citrate-based sponge 601 may be placed on the button suture loop 107 in the fibular tunnel 507 between the button 103 and the bone anchor 101. When the button suture loop 107 is tightened, the sponge 601 may fill the fibular tunnel 507 and extrude beyond the fibula medial cortex into the interosseous space between the fibula 503 and tibia 501 eluting citrate to aid in healing the damaged syndesmotic membrane.

At 711, the ends 113 of the button suture loop 107 are pulled, tightening the button suture loop 107 and biasing the button 103 towards the bone anchor 101. As the button suture loop 107 is tightened, the button 103 is pulled into the fibular tunnel 507. The end 113 of the button suture loop 107 is pulled until a desired tension between the bone anchor 101 and the button 103 is achieved, i.e., tension between the tibia 501 and the fibula 503. In some embodiments, it may be desirable to fix a high ankle sprain with a tension force of between 40 to 50 Newtons. In some embodiments, the syndesmosis fixation device may be rated to apply a tension force of upwards of 180 Newtons. In some embodiments, the syndesmosis fixation device may be rated to apply a tension force of upwards of 400 Newtons. The specific limits for a given syndesmosis fixation device depends on the bone anchor 101, button 103, sutures, and coupling configuration used. For example, when the suture loops 105, 107 are in a double looped configuration, the tension force is distributed over four suture strands allowing.

At 713, once the button suture loop 107 is tightened to a desired tension, the ends 113 of the button suture loop 107 may be trimmed or cut off. In some embodiments, the ends of the button suture loop 107 may be tied off to provide additional secure fixation. The procedure may be performed as many times as desired to insert additional syndesmosis fixation devices 100 to facilitate healing the syndesmosis injury. It should be appreciated that while the procedure may be repeated as many times as desired the multiple procedures may be carried out in parallel. For example, if two syndesmosis fixation devices 100 are being installed, the bone tunnels for both devices may be drilled before the first syndesmosis fixation device 100 is installed.

In some embodiments, the bone anchor 101 and the button 103 are made of a citrate-based polymer which both maintains mechanical stability between the tibia 501 and the fibula 503, allowing the polymer composite to integrate and metabolize into the patient's own bone while also participating in the soft tissue healing phase of the syndesmosis.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments described herein.

While the disclosure is provided in detail in connection with only a limited number of embodiments, it should be readily understood that the disclosure is not limited to such disclosed embodiments. Rather, the disclosure can be modified to incorporate any number of variations, alterations, substitutions or equivalent arrangements not heretofore described, but which are commensurate with the spirit and scope of the disclosure. Additionally, while various embodiments of the disclosure have been described, it is to be understood that the exemplary embodiment(s) may include only some of the described exemplary aspects. Accordingly, the disclosure is not to be seen as limited by the foregoing description but is only limited by the scope of the appended claims.

1. A syndesmosis fixation system comprising:
a button;
a bone anchor having an insertion end configured to be inserted into a bone tunnel, the bone anchor having one or more barbs configured to prevent the bone anchor from being dislodged once inserted into the bone tunnel;
a first suture loop coupled to the insertion end of the bone anchor; and
a second suture loop coupled to the first suture loop and coupled to the button;
wherein the first suture loop and the second suture loop operatively connect the button to the bone anchor;
wherein the second suture loop is actuatable to tighten the second suture loop, biasing the button toward the bone anchor.

2. The system of claim 1, wherein the button and the bone anchor are made, in whole or in part, from a citrate-based polymer composite.

3. The system of claim 1, wherein the bone anchor defines a hole opposite the insertion end, the hole being sized and dimensioned to receive at least part of an inserter to facilitate insertion of the bone anchor into the bone tunnel.

4. The system of claim 1, wherein the second suture loop is a self-locking cinch.

5. The system of claim 1, wherein the one or more barbs are a single spiral barb, a double concentric spiral barb, a double alternating spiral barb, a circular ring barb, or a diamond shaped barb.

6. The system of claim 1, further comprising a first sleeve disposed around at least a portion of the first suture loop and a second sleeve disposed around at least a portion of the second suture loop.

7. The system of claim 6, wherein the first sleeve and the second sleeve reinforce a point of contact between the first suture loop and the second suture loop.

8. The system of claim 1, further comprising a citrate-based synthetic sponge disposed in the second suture loop between the button and the bone anchor.

9. The system of claim 8, wherein the citrate-based synthetic sponge contains a citrate based solution.

10. The system of claim 1, wherein the button is a trauma plate.

11. A syndesmosis fixation system comprising:
a button;
a bone anchor having an insertion end configured to be inserted into a bone tunnel, the bone anchor having one or more barbs configured to prevent the bone anchor from being dislodged once inserted into the bone tunnel;
a suture loop operatively connecting the button to the bone anchor, the suture loop being coupled to an end of the bone anchor opposite the insertion end of the bone anchor;
wherein the suture loop is actuatable to tighten the suture loop, biasing the button toward the bone anchor.

12. The system of claim 11, wherein the button and the bone anchor are made, in whole or in part, from a citrate-based polymer.

13. The system of claim 11, wherein the button is a trauma plate.

14. A method for performing syndesmosis fixation comprising:
anatomically aligning a fibula and a tibia of a patient into a position to be fixed;
drilling a fibular tunnel through the fibula;
drilling a tibial tunnel into the tibia concentric with the fibular tunnel;
attaching a bone anchor of a syndesmosis fixation system to an inserter, the syndesmosis fixation system comprising:
a button;
an anchor:
the bone anchor having an insertion end configured to be inserted into the tibial tunnel, the bone anchor having one or more barbs configured to prevent the bone anchor from being removed once inserted into the tibial tunnel;
one or more suture loops operatively connecting the button to the bone anchor;
wherein at least one of the one or more suture loops is actuatable to tighten at least one suture loop, biasing the button toward the bone anchor;
inserting the bone anchor through the fibular tunnel and into the tibial tunnel;
detaching the inserter from the bone anchor;
actuating at least one of the one or more suture loops to fix the fibula and the tibia together by biasing the button towards the bone anchor and against the fibula lateral cortex.

15. The method of claim 14, wherein a jig is used when aligning the fibula and the tibia of the patient into the position to be fixed.

16. The method of claim 14, further comprising inserting a citrate-based synthetic sponge into the fibular tunnel after detaching the inserter from the bone anchor and before actuating at least one of the one or more suture loops.

17. The method of claim 16, wherein when the button is biased against the later cortex of the fibula or fibular plate, the sponge extends past the fibula medial cortex into an interosseous space between the tibia and the fibula.

18. The method of claim 17, wherein the citrate-based synthetic sponge retains a citrate composition to aid in healing a syndesmotic membrane.

19. The method of claim 14, further comprising drilling a counter bored hole in the fibula lateral cortex concentric with the fibular tunnel.

20. The method of claim 19, wherein when the button is biased against the fibula lateral cortex, the button sits in the counter bored hole flush with the fibula.

21. The method of claim 20, wherein the fibula tunnel, the tibial tunnel, and the counter bored hole are drilled using a single drill bit having a variable diameter.

22. The method of claim 14, wherein the button of the syndesmosis fixation device is a trauma plate.
